(19) United States
(12) Patent Application Publication
(10) Pub. No.: US 2020/0129326 A1
(43) Pub. Date: Apr. 30, 2020

(54) A Blanket for Forced Air Warming of a Patient

(71) Applicant: Care Essentials Pty Ltd, Victoria (AU)

(72) Inventors: Abhay Sinha, Victoria (AU); Ishan Sinha, Victoria (AU)

(73) Assignee: Care Essentials Pty Ltd, Victoria (AU)

(21) Appl. No.: 16/606,855

(22) PCT Filed: Apr. 27, 2018
(86) PCT No.: PCT/AU2018/050386

§ 371 Date: Oct. 21, 2019

(30) Foreign Application Priority Data
May 2, 2017 (AU) 2017901597

(51) Int. Cl. A61F 7/00

(52) U.S. Cl. CPC A61F 7/0097; A61F 2007/0091; A61F 2007/0029; A61F 2007/0018; A61F 2007/0022; A61F 2007/0039; A61F 2007/0055; A61F 2007/002

(57) ABSTRACT

A blanket for forced air warming of a patient, the blanket including a non-air permeable outer layer an air permeable inner layer contactable to the patient; the outer layer and inner layer defining an inflatable chamber, the inflatable chamber allowing egress of the forced warming air through the inner layer; wherein in use, the inflated chamber provides visual transparency of the patient.

### FIELD OF THE INVENTION

The present invention relates to a blanket for forced air warming of a patient, used in the treatment of patients to maintain a patient's core body temperature as is required before, during and after medical surgery.

### BACKGROUND OF THE INVENTION

Conventionally, medical clinicians, and nurses need to observe and monitor the body and specific body parts of the patients under anaesthesia, whilst the patient is being warmed pre-operation, during an operation and post-operation, to avoid any potential medical complications and to enable timely medical intervention, if required. Clinicians are very careful about observing and monitoring the body and the body parts of patients, including different pressure points, whilst the patient is under general anaesthesia.

In addition, there are tubes (Including intravenous tubes, blood circulation tubes and oxygen tubes) running from the body of patients to external medical equipment during and after the operation and surgeons, clinicians, anaesthetists must keep an expert eye on this tubing on a continual basis.

Patient warming has become an important component before, during and after surgery. Increased throughput and efficiency may be attained by hospitals in their post-operative recovery care facilities as a result of using patient warning protocols.

Supplying forced warm filtered air, blown directly onto the patient has resulted in significantly improved control of patient warning. Patient core temperatures increases have been achieved using warmed filtered air blown through warming blankets. The use of force warmed filtered air provides a number of benefits including increased blood flow, improved patient comfort, reduced recovery time in post-operative care and reduction in post-operative infection.

The problem of using the currently available forced air warming medical blankets in the market is that they visually block and the make the patient opaque from the clinician. The clinician must physically turn the blanket up, in order to observe the patient's body and the body parts of the patient, whilst the patient is under anaesthesia. This can be problematic, as the anaesthetists also must contemporaneously perform other tasks. Importantly, the effectiveness of patient warming is compromised every time the clinician lifts the current forced air warming blankets to observe the patient, as the patient can quickly cool down its core body temperature whilst under anaesthesia.

It therefore an object of this invention to provide for a forced air warming medical blanket, for clinicians, surgeons, anaesthetists and nurses, to continuously and visually observe the patient's body parts, pressure points and connected medical tubing and ameliorate all the above associated problems.

### SUMMARY OF INVENTION

Accordingly, the present invention provides a blanket for forced air warming of a patient, the blanket including:
a non-air permeable transparent outer layer;
an air permeable transparent inner layer contactable to the patient;
the outer layer and inner layer being joined at the periphery of the inner layer to define an inflatable chamber, the chamber allowing egress of the forced warming air through the inner layer;
wherein in use, the inflated chamber provides visual transparency of the patient.
It is preferred that the outer layer and the inner layer are both constructed from biaxially orientated polypropylene film
It is preferred that the outer layer and the inner layer are both constructed from any one or more of the following: biaxially orientated polypropylene film; polypropylene film; polyvinyl chloride; and other transparent medical grade plastic.
It is preferred that the inner layer includes an array of perforations.

One advantage of the blanket for forced air warming of a patient is that medical clinicians and anaesthetists can make continuous visual medical observations of the patient's body parts and pressure points through the blanket, without the need to lift the blanket.

Another advantage of the blanket for forced air warming of a patient is that medical clinicians and anaesthetists can make continuous visual medical observations of the patient's tubing to various medical equipment through the blanket, without the need to lift the blanket.

It will be convenient to hereinafter to describe the invention with reference to the following drawings which shows ten (10) preferred embodiments of the blanket for forced air warming of a patient, according to the invention. The particularity of the attached drawings and following description should not supersede or limit the preceding broad definition of the invention.

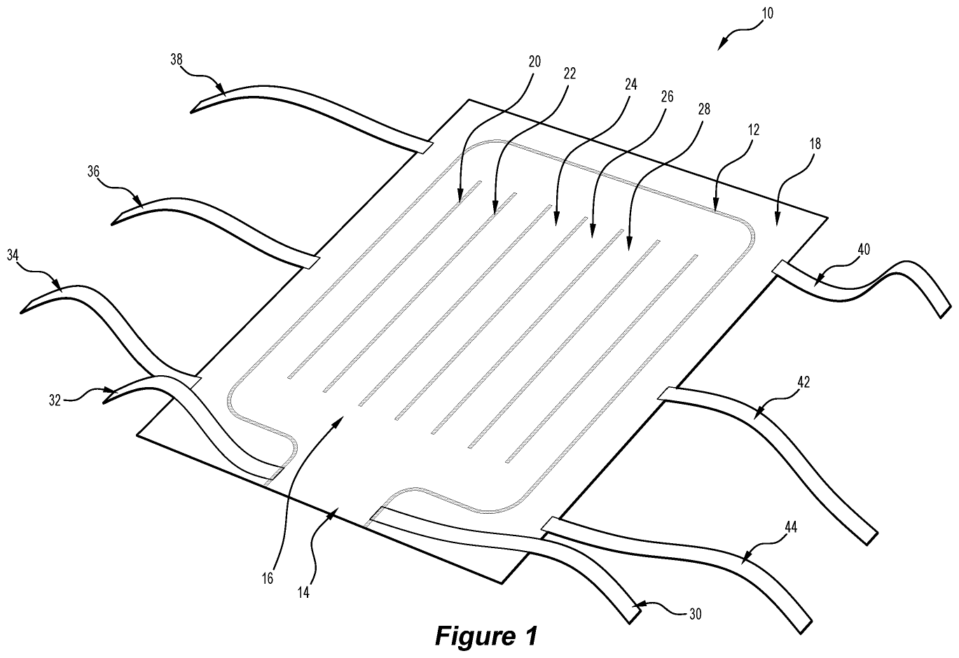

FIG. 1, is a perspective view of the blanket for forced air warming of a patient according to a first preferred embodiment of the invention, illustrating the blanket in an unassembled state before use on the patient;

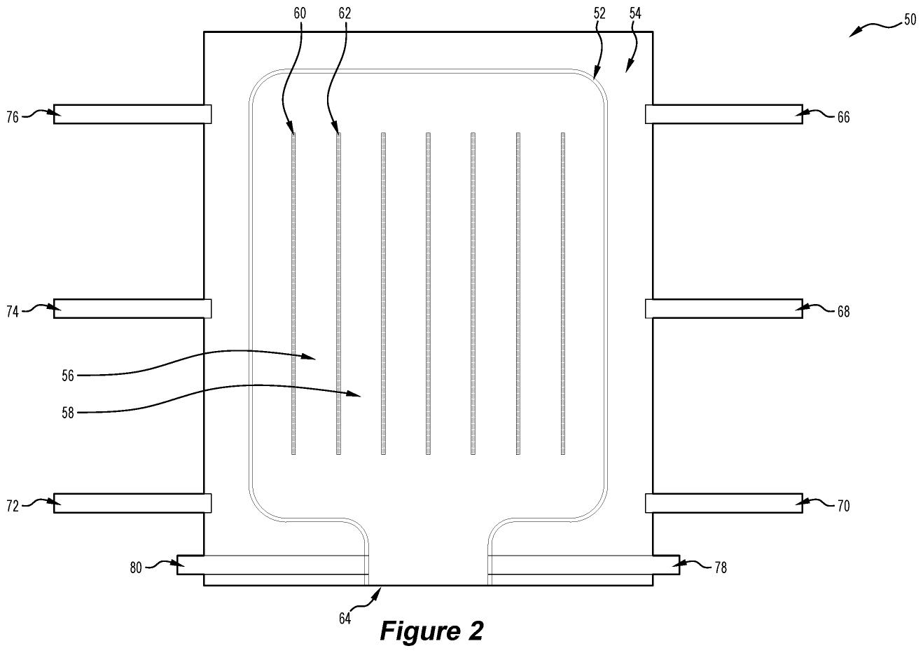

FIG. 2 is a top perspective view of the blanket for forced air warming of a patient of FIG. 1, according to a second preferred embodiment of the invention, illustrating the blanket in an unassembled state before use on the patient and the facing outer layer;

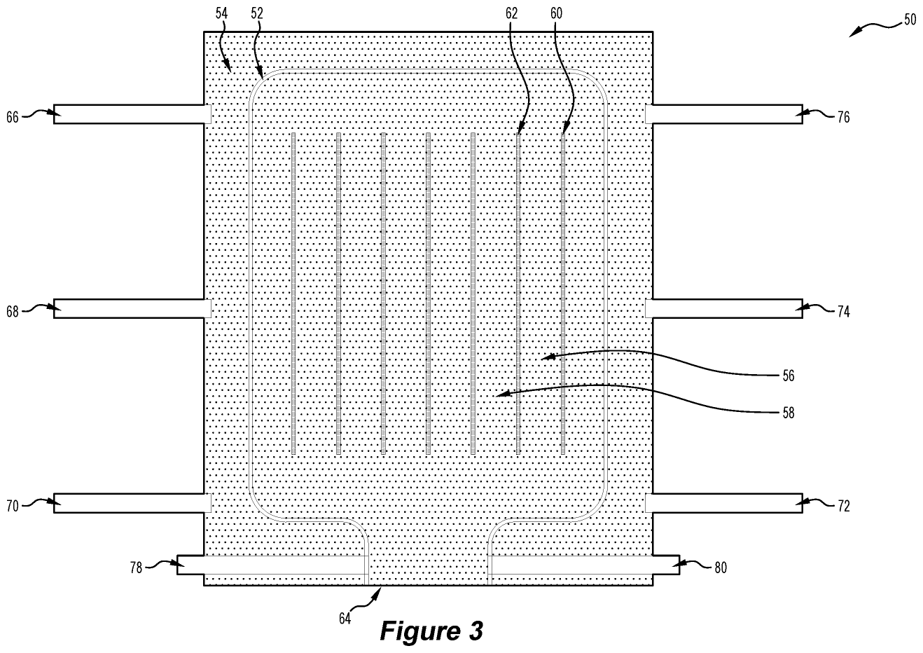

FIG. 3 is a bottom perspective view of the blanket for forced air warming of a patient of FIG. 1, according to a third preferred embodiment of the invention, illustrating the blanket in an unassembled state before use on the patient and the facing inner layer;

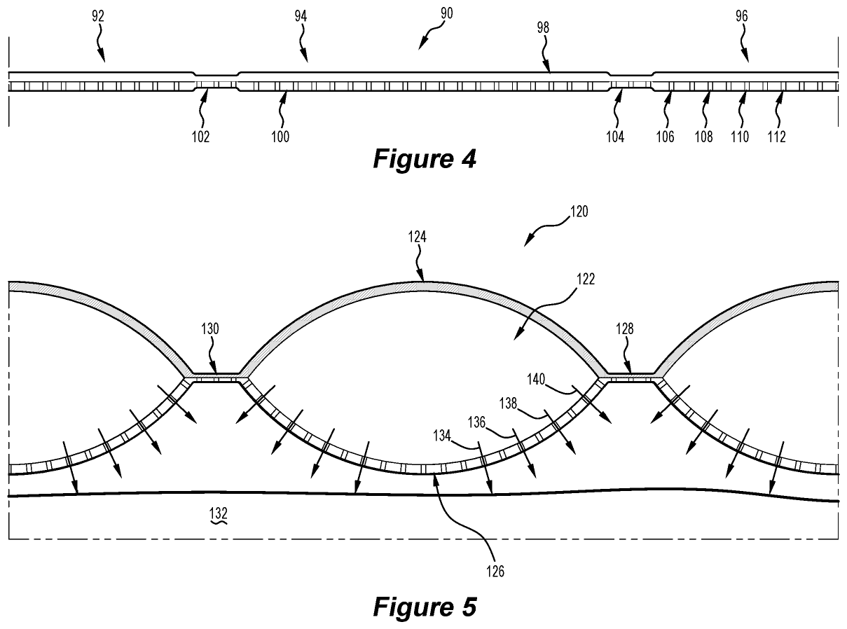

FIG. 4 is a cross sectional view of an inflatable chamber of the blanket for forced air warming of a patient of FIG. 1, according to a fourth preferred embodiment of the invention, illustrating the inflatable chamber with no forced warming air, passing therethrough;

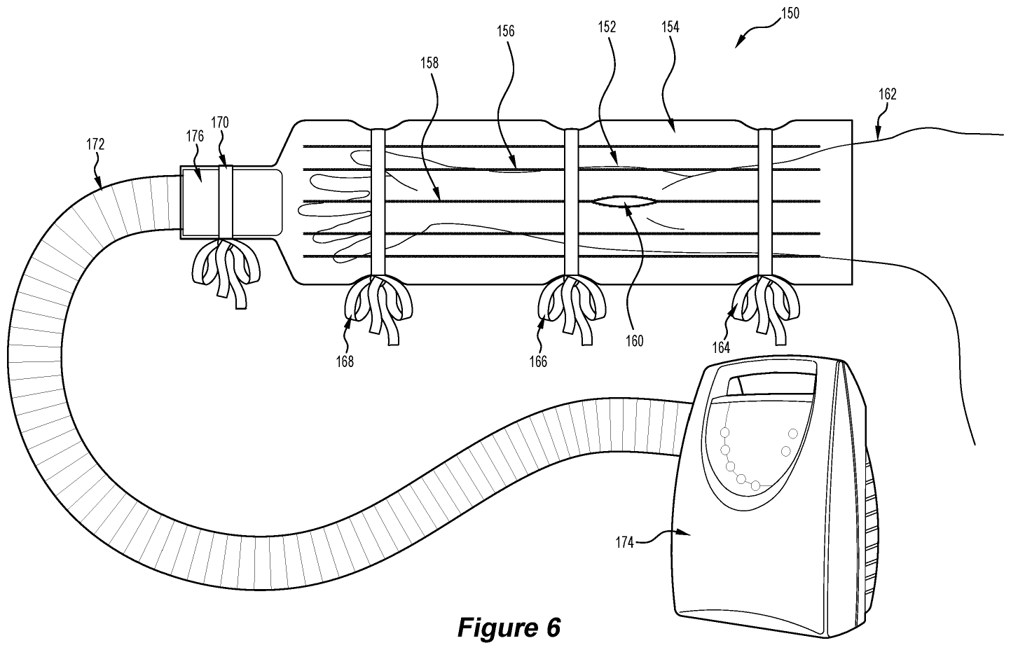

FIG. 5 is a cross sectional view of the inflatable chamber of FIG. 4, according to a fifth preferred embodiment of the invention, illustrating the inflatable chamber in an expanded position egressing forced warming air through the inner layer and onto the patient;

FIG. 6 is a perspective view of the blanket for forced air warming of a patient of FIG. 1, used on top of the patient's arm and being connected to a forced warming machine and connectable hose, according to a sixth preferred embodiment of the invention;

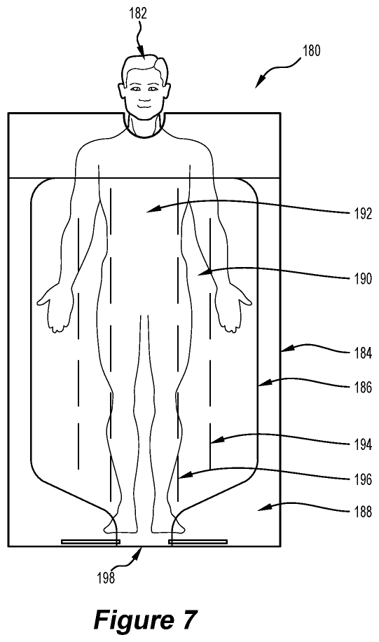

FIG. 7, is a top perspective view of an alternative embodiment of the blanket for forced air warming of a patient, according to a seventh preferred embodiment of the invention, illustrating the full-bodied blanket and the transparency of the patient;

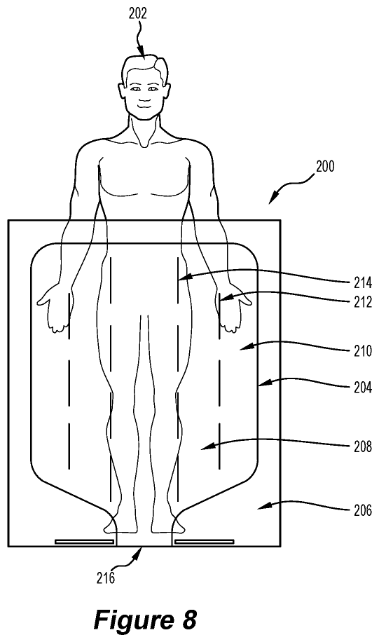

FIG. 8, is a top perspective view of a further alternative embodiment of the blanket for forced air warming of a patient, according to an eighth preferred embodiment of the invention, illustrating the lower bodied blanket and the transparency of the patient;

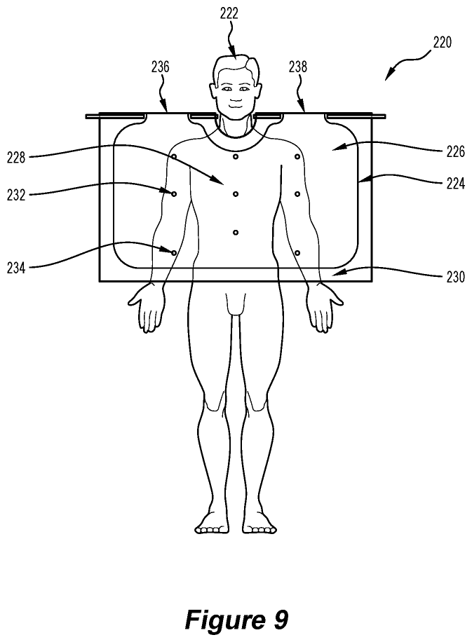

FIG. 9 is a top perspective view of a further alternative embodiment of the blanket for forced air warming of a patient, according to a ninth preferred embodiment of the invention, illustrating the upper body arms-in blanket and the transparency of the patient;

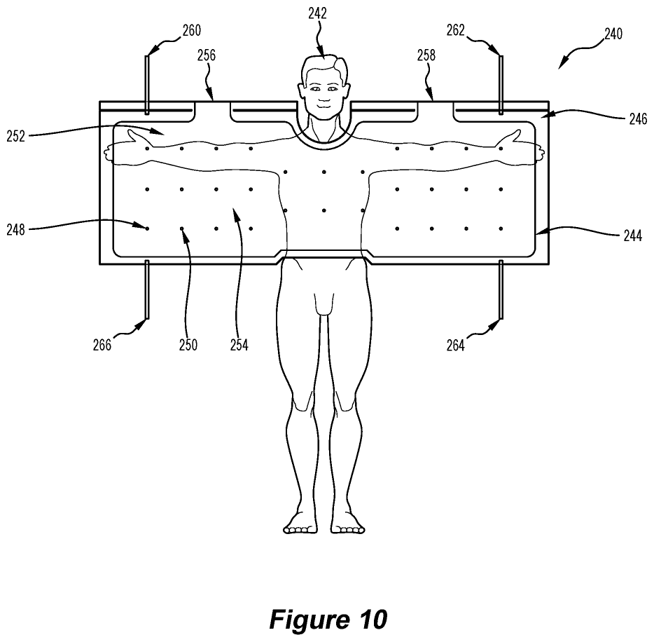

FIG. 10 is a perspective view of the further alternative embodiment of the blanket for forced air warming of a patient, according to a tenth preferred embodiment of the invention, illustrating the upper body arms out blanket and the transparency of the patient;

### DETAILED DESCRIPTION OF THE INVENTION

FIG. 1, is a perspective view of the blanket for forced air warming of a patient 10, illustrating the blanket in an unassembled state before use on the patient, is provided with an inflatable chamber 12. The inflatable chamber 12 is constructed by an outer layer and inner layer being joined at the periphery of the inner layer. The person skilled in the art would appreciate that the inflatable chamber 12 extends not at the absolute outer edge of the inner layer but its periphery. The outer layer is non-air permeable and the inner layer is air permeable. The outer layer presents visually upside toward the medical clinician when the blanket for forced air warming of a patient 10 is placed on the patient. The air permeable inner layer is contactable with the patient when the blanket for forced air warming of a patient 10 is used. The inflatable chamber 12 is inflatable when receiving forced warming air from any convenient air warming machine currently in use (not shown) though an air inlet 14, the forced warming air which extends into inflatable chamber space 16 that expands the inflatable chamber 12 from its collapsed position.

The non-inflatable portion 18 is not functional in the sense that it does not form part of the inflatable chamber 12, but it is defined by the loose association of the outer layer and inner layer.

The inflatable chamber 12 is defined by joining of the outer layer and periphery of the inner layer or integrally forming of the outer layer and the inner layer of the periphery of the inner layer or thermal welding of the outer layer and inner layer at the periphery of the inner layer.

The blanket for forced air warming of a patient 10 is transparent. That is, the user of the blanket for forced air warming of a patient 10 being the clinician and/or medical nurse advantageously can visually inspect and see through the blanket and the entire limbs of the patient that is covered by the blanket for forced air warming of a patient 10. Each of the outer layer and the inner layer are constructed from any one or more of the following: biaxially orientated polypropylene film, polypropylene film, polyvinyl chloride (PVC) and other medical grade plastic material having a transparent function. It must be stated that biaxially orientated polypropylene film is benign on the human skin and the ideal material for such blankets.

The air permeable inner layer is constructed with plurality or an array of small apertures or perforations (not shown in FIG. 1) to provide an egress function of the forced warming air from inside the inflatable chamber 12 through the air permeable inner layer and onto the patient for warming to the appropriate body temperature as determined by the clinician and/or nurse. The perforations are evenly spread across the entire surface area of the inner layer such that they operatively apply as a whole, continuous forced air warming over the relevant surface area of the patient. The perforations are of any shape including annular, spherical and square shape.

The inflatable chamber 12 consists of a plurality of interconnected compartments 24, 26 & 28. Specifically, interconnected compartments 24, 26 & 28 which are all disposed or positioned in parallel with each other and are fluted. The person skilled in the art would appreciate that the interconnected compartments 24, 26 & 28 are functionally connected to each other at their respective ends. These interconnected compartments 24, 26 & 28 are formed or defined by joining of a portion of the outer layer and a corresponding portion of the inner layer, or integrally forming of a portion of the outer layer and a corresponding portion of the inner layer or thermal welding of a portion of the outer layer and a corresponding portion of the inner layer to define join 20 and join 22. Alternatively, the join 20 and join 22 is also formed by way of ultrasonic welding a portion of the outer layer and a corresponding portion of the inner layer. Advantageously, the interconnected compartments 24, 26 & 28 functions to provide a uniform and longitudinal distribution of forced warming air over a patient.

Machine ties 30 and 32 serve the function of securing the blanket for forced air warming of a patient 10 to a hose of the forced air warming machine (both the hose and forced air warming machine not shown in FIG. 1). The machine ties 30 and 32 are constructed from any suitable material, including cotton, nylon, Velcro™ and any other suitable material. Patient ties 34, 36, 38, 40, 42 and 44, serve the function of securing the blanket for forced air warming of a patient 10 to the patient after the blanket for forced air warming of a patient 10 is wrapped around the arm or body of the patient (both not shown in FIG. 1). The patient ties 34, 36, 38, 40, 42 and 44 are constructed from any suitable material, including cotton, nylon, Velcro™ and any other suitable material.

Turning to FIG. 2, being the top perspective view of the blanket for forced air warming of a patient 50, having the inflatable chamber 52, the inflatable chamber 52 in turn, includes a plurality of interconnected compartments 56 and 58 separated by join 60 and join 62 respectively. The non-inflatable portion 54 is defined by the loose association of the outer layer and inner layer.

The join 60 and join 62 are created by joining a portion of the outer layer and a corresponding portion of the inner layer, or integrally forming of a portion of the outer layer and a corresponding portion of the inner layer or thermal welding of a portion of the outer layer and a corresponding portion of the inner layer. Alternatively, the join 60 and join 62 are each formed by way of ultrasonic welding a portion of the outer layer and a corresponding portion of the inner layer.

Air inlet 64 extends into or articulates with the inflatable chamber 52 such that when forced warming air is blown through the air inlet 64 and into the inflatable chamber 52 when the blanket for forced air warming of a patient 50 is in a lay-flat orientation and the forced warming air is blown substantially parallel with the interconnected compartment 56 and interconnected compartment 58.

Patient ties 66, 68, 70, 72, 74 and 76 serve the function of securing the blanket for forced air warming of a patient 50 to the patient (not shown) after the blanket for forced air warming of a patient 50 is wrapped around the arm or body of the patient (both not shown in FIG. 2). Machine tie 78 and machine tie 80 serve the function of securing the blanket for forced air warming of a patient 50 to a hose of the forced air warming machine (both not shown in FIG. 2).

Turning to FIG. 3 is a bottom perspective view of the blanket for forced air warming of a patient 50, illustrating the blanket for forced air warming of a patient 50 in an unassembled state before use on the patient and the facing inner layer. The blanket for forced air warming of a patient 50 has the inflatable chamber 52 and a plurality of interconnected compartments 56 and 58 therethrough, separated by join 60 and join 62 The non-inflatable portion 54 is defined by the loose association of the outer layer and inner layer. The joins 60 and join 62 are created by joining a portion of the outer layer and a corresponding portion of the inner layer, or integrally forming of a portion of the outer layer and a corresponding portion of the inner layer or thermal welding of a portion of the outer layer and a corresponding portion of the inner layer. Alternatively, the join 60 and join 62 is also formed by way of ultrasonic welding a portion of the outer layer and a corresponding portion of the inner layer.

The air inlet 64 extends into or articulates with the inflatable chamber 52 such that when forced warming air is blown through the air inlet 64 and into the inflatable chamber 52 when the blanket for forced air warming of a patient 50 is in a lay-flat orientation and the forced warming air is blown substantially parallel with the interconnected compartment 56 and interconnected compartment 58.

Patient ties 66, 68, 70, 72, 74 and 76 serve the function of securing the blanket for forced air warming of a patient 50 to the patient (not shown) after the blanket for forced air warming of a patient 50 is wrapped around the arm or body of the patient (both not shown in FIG. 3). Machine tie 78 and machine tie 80 serve the function of securing the blanket for forced air warming of a patient 50 to a hose of the forced air warming machine (both not shown in FIG. 3).

The person skilled in the art would appreciate by considering FIG. 3. that the inner layer has spotted shading, which illustrates the perforations of the inner layer. These perforations provide the necessary egress function of the forced warm air to pass through the inflatable chamber 52 and through the perforations defined within which is further described by FIG. 4 below.

Turning to FIG. 4 is a cross sectional view of the inflatable chamber 90 of the blanket for forced air warming of a patient of FIG. 1, illustrating the interconnected compartments 92, 94 and 96 all in a flat and un-used state with no forced warming air passing therethrough. The outer layer 98 is flexible and impermeable to air engages the inner layer 100 which is also flexible and is air permeable. Perforations 106, 108, 110 and 112 disposed within the inner layer 100 provide respective air channels through the entire inner layer 100. The join 102 and join 104 are each defined by joining a portion of the outer layer 98 and a corresponding portion of the inner layer 100, or integrally forming of a portion of the outer layer 98 and a corresponding portion of the inner layer 100 or thermal welding or ultrasonic welding of a portion of the outer layer 98 and a corresponding portion of the inner layer 100.

Turning to FIG. 5 is the cross-sectional view of the inflatable chamber 120 of FIG. 4 illustrating the inflatable chamber 120 in an expanded position egressing forced warming air through the inner layer 126 and onto a patient 132. The interconnected compartment 122 has flexibly expanded by way of forced warming air passing therethrough. The outer layer 124 being flexible and impermeable to air, flexibly expands as well as the inner layer 126 which is also flexible, but is air permeable. Egressed air 134, 136, 138 and 140 through corresponding perforations (unnumbered) disposed within the inner layer 126 provide the air channel through the entire inner layer 126 and onto the patient 132 (in part) including a dorsum hand or arm of the patient 132. The join 128 and join 130 are each sufficiently bonded or joined together to provide the necessary structure of the interconnected compartment 122 during the forced warming air egressed state.

By considering FIG. 3, FIG. 4 and FIG. 5, the person skilled in the art would appreciate that the inner layer has an array of perforations throughout the entire surface area of the inflatable chambers 52, 90 and 120 respectively, which provides the necessary egress function of the forced warming air through the inner layer 100 of FIG. 4 and inner layer 126 of FIG. 5 and onto the patient 132 of FIG. 5.

Now FIG. 6 is a perspective view of the blanket for forced air warming of a patient 150 used on top of the patient's arm 162 and being connected to the forced air warming machine 174 and hose 172 which channels the forced warming air to the blanket for forced air warming of a patient 150 through the air inlet 176 and through the interconnected compartment 152 and interconnected compartment 154 situated parallel with each other and are fluted. The join 156 and join 158 separate the interconnected compartments 152 and 154 which are previously described herein.

The person skilled in the art will appreciate that in use, the interconnected compartment 152 and interconnected compartment 154 are disposed substantially coaxially with the patient's arm 162 when the blanket for forced air warming of a patient 150 is used. The person skilled in the art will also appreciate that FIG. 6 illustrates the transparency of the blanket for forced air warming of a patient 150 such that the patient's arm 162 is clearly transparent through the blanket for forced air warming of a patient 150, at all times during use by the clinician or medical nurse (not illustrated in this FIG. 6). The person skilled in the art will further appreciate that FIG. 6 illustrates that the blanket for forced air warming of a patient 150 in use is placed on top of the patient's arm 162. This means that in use, the blanket for forced air warming of a patient 150 can be lifted up momentarily above the dorsal hand near the air inlet 176 such the dorsal hand can be accessed for cannulation by the medical practitioner of the patient's veins, being the dorsal metacarpal veins, dorsal venous plexus, cephalic vein and basilic vein.

The blanket for forced air warming of a patient 150 optionally includes an access port 160 which allows the clinician, anaesthetist and nurse to visually identity and physically access the patient's arm 162 and perform medical treatment (including venipuncture or the administration of cannula) on the patient's arm 162 through the access port 160. The patient's arm 162 is positioned or disposed adjacent or situated in a manner whereby the patient's cubital fossa and therefore median cubital vein is accessible through the access port 160. The transparency of the blanket for forced air warming of a patient 150 is enhanced by the access port 160 even though the access port 160 is optional. Other patient's veins are also accessible near the cubital fossa, include the cephalic vein and the basilic vein. The patient ties 164, 166 and 168 are illustrated in use which are assembled by hand tying by the clinician, anaesthetist and nurse when assembling or securing the blanket for forced air warming of a patient 150 to the patient's arm 162.

In normal use, the blanket for forced air warming of a patient 150 can be used in a variety of ways. Firstly, the blanket for forced air warming of a patient 150 is wrapped around the patient's arm 162 and tied using the patient ties 164, 166 and 168 by the clinician's hands (not shown). The hose 172 of the forced air warming machine 174, is then inserted into the air inlet 176 and tied together by the machine tie 170, by tying over the air inlet 176 end of the blanket for forced air warming of a patient 150. The forced air warming machine 174 is then turned on by the clinician or nurse and then continuous forced warming air then passes through the hose 172 and the air inlet 176 and expands the inflatable chamber (not numbered) by forced warming air passing through the interconnected compartment 152 and interconnected compartment 154 and further through the inner layer (not numbered) as described above and warming the patient's arm 162 with continuous forced warming air. Secondly and alternatively, the blanket for forced air warming of a patient 150 is placed on top of the patient's arm 162 without the need for using the patient tie 164, patient tie 166 and patient tie 168.

FIG. 7, is a top perspective view of an alternative embodiment of the blanket for forced air warming of a patient 180, illustrating the full-bodied blanket and the transparency of the patient 182 therethrough. The blanket edge 184 includes a non-inflatable portion 188, and the inflatable chamber 186 is defined in the manner described herein. The interconnected compartments 190 and 192 are fluted and separated by join 194 and join 196 (defined and formed which is described herein) and are disposed in parallel with each other and in this embodiment, are coaxial with the torso and/or legs of the patient 182 as a whole when used. The air inlet 198 allows forced warming air (not illustrated) to enter the blanket for forced air warming 180 and warm the entire patient 182 from neck to toe.

FIG. 8, is a top perspective view of a further alternative embodiment of the blanket for forced air warming of a patient 200, illustrating the lower bodied blanket and the unique transparency of the patient 202 therethrough. The blanket for forced air warming of a patient 200 includes a non-inflatable portion 206 and the inflatable chamber 204 defined in the manner described above. The interconnected compartments 208 and 210 are fluted and separated by joins 212 and 214 (defined and formed which is described herein) and are disposed in parallel with each other and in this embodiment, are coaxial with the torso and/or legs of the patient 202 as a whole, when used. The air inlet 216 allows forced air warming (not illustrated) to enter the blanket for forced air warming of a patient 200 thereby warming the lower body of the patient 202 before, during and after medical surgery.

FIG. 9 is a top perspective view of a further alternative embodiment of the blanket for forced air warming of a patient 220 illustrating the upper body arms-in blanket and the transparency of the patient 222 therethrough. The blanket for forced air warming of a patient 220 includes a non-inflatable portion 230 and the inflatable chamber 224 is defined in the same manner described herein.

The inflatable chamber 224 includes interconnected compartments 226 and 228 that are different to FIG. 1 to FIG. 8 in that the joins 232 and 234 are formed by joining an annular portion of the outer layer and a corresponding annular portion of the inner layer or integrally forming of an annular portion of the outer layer and a corresponding annular portion of the inner layer or thermal welding or ultrasonically welding of an annular portion of the outer layer and a corresponding annular portion of the inner layer. The interconnected compartments 226 and 228 are in more open communication with each other in this embodiment of the inflatable chamber 224, as compared to FIG. 1 to FIG. 8 having interconnected compartments disposed in parallel with each other. Each air inlet 236 and 238 allows for the forced warming air (not illustrated) to enter the blanket for forced air warming of a patient 220 thereby warming the upper body and arms of the patient 222.

Although the blanket for forced air warming of a patient 220 of FIG. 9 has an annular join 232 and annular join 234 described above, an alternative embodiment of FIG. 9, includes interconnected compartments which are all disposed in parallel and fluted similar to FIG. 1 to FIG. 8.

FIG. 10, is a top perspective view of the further alternative embodiment of the blanket for forced air warming of a patient 240 illustrating the upper body arms-out blanket and the transparency of the patient 242. The blanket for forced air warming of a patient 240 includes a non-inflatable portion 246 and the inflatable chamber 244 is defined in the manner described below.

The inflatable chamber 244 includes interconnected compartments 252 and 254 that are different to FIG. 1 to FIG. 8 in that the join 248 and join 250 are formed by joining an annular portion of the outer layer and a corresponding annular portion of the inner layer or integrally forming of an annular portion of the outer layer and a corresponding annular portion of the inner layer or thermal welding or ultrasonically welding of an annular portion of the outer layer and a corresponding annular portion of the inner layer. The interconnected compartments 252 and 254 are more openly communicated with each other in this embodiment of the inflatable chamber 244 as compared to FIG. 1 to FIG. 8. Each air inlet 256 and 258 allows for the forced warming air (not illustrated) to enter the blanket for forced air warming of a patient 240 thereby warming the upper body and arms of the patient 242. Patient ties 260, 262, 264 and 266 are illustrated but not used in this FIG. 10 but are used to secure the blanket for forced air warming of a patient 240 around the arms of the patient 242 in a crucifix type position and keeping the arms and chest of the patient warm by forced air warming.

Although the blanket for forced air warming of a patient 240 of FIG. 10 has an annular join 248 and annular join 250 described above, an alternative embodiment of FIG. 10, includes interconnected compartments which are all disposed in parallel and fluted similar to FIG. 1 to FIG. 8.

All of the above embodiments FIG. 1 to FIG. 10 of the blanket for forced air warming of a patient 10, 50, 150, 180, 200, 220 and 240 are single use disposable blankets. Once the blanket for forced air warming of a patient 10, 50, 150, 180, 200, 220 and 240 are used on a single patient they are destroyed so as to avoid any potential cross-infection by using on subsequent patients. This reduces cleaning and laundry costs for medical clinics and hospitals.

It will also be appreciated by the person skilled in the art that the blanket for forced air warming of a patient 10, 50, 150, 180, 200, 220 and 240 are still functional even without using the relevant patient ties 34, 36, 38 40, 42 and 44 (of FIG. 1 as a non-limiting example only). The blanket for forced air warming of a patient 10 can be wrapped under the patient's arm, with no need for tying of the patient ties 34, 36, 38 40, 42 and 44. The blanket for forced air warming of a patient 10 can still be inflated providing forced warming air to the patient's arm.

The unique transparency of the blanket for forced air warming of a patient 10, 50, 150, 180, 200, 220 and 240 provides the advantage by reducing the need for the surgeons, clinicians, anaesthetists to undo the hand ties 34, 36, 38 40, 42 and 44 (of FIG. 1 as a non-limiting example only) in order to lift the blanket for forced air warming of a patient 10, 50, 150, 180, 200, 220 and 240 to make visual medical observations.

Although the blanket for forced air warming of a patient 10, 50, 150, 180, 200, 220 and 240 are described herein for blankets, the principles used for blankets is used as patient gowns for use by patient's in hospitals using the forced air warming machine and uniquely designed medical gown. The person skilled in the art would appreciate that a variation on FIG. 7 contemplates a patient's gown.

The unique visual transparency function for viewing the patients through the blanket for forced air warming of a patient 10, 50, 150, 180, 200, 220 and 240 of FIG. 1 to FIG. 10 whilst undertaking forced air warming was developed by significant trial and error of the Applicant. The unique outer layer and the inner layer are both biaxially orientated polypropylene film which provides the unique transparency function.

The use of biaxially orientated polypropylene film resulted from identifying the initial problem of the prior art, which uses a non-woven fabric and non-transparent material being polypropylene spun bond material. The Applicant then commenced a trial to first move from the existing prior art non-woven fabric to a non-woven and transparent fabric. The transparency could not be achieved as the prior art fabric was a non-woven fabric.

Subsequently, biaxially orientated polypropylene film was then uniquely chosen by the Applicant as it is inert to human patient's skin. Because of this trial and error, the unique result of transparency was achieved using biaxially orientated polypropylene film for the outer layer and inner layer. Other materials such as polyvinyl chloride (PVC) and other medical grade plastic material having a transparent function can also be used.

One advantage of the blanket for forced air warming of a patient is that medical clinicians and anaesthetists can make continuous visual medical observations of the patient's body parts and pressure points through the blanket, without the need to lift the blanket.

Another advantage of the blanket for forced air warming of a patient is that medical clinicians and anaesthetists can make continuous visual medical observations of the patient's tubing to various medical equipment through the blanket, without the need to lift the blanket.

Various alterations and/or additions may be made to the blanket for forced air warming of a patient hereinbefore described in this Specification, without departing from the spirit, ambit or scope of the invention. The person skilled in the art would appreciate that a variation to the blanket for forced air warming of a patient includes many different blankets having any suitable shape for medical purposes including a paediatric lower body blanket, a cardiac access blanket, abdominal access blanket, multi-access blanket, surgical access-arms in blanket, and a lithotomy and laparoscopy blanket.

Further, the person skilled in the art would appreciate that a variation on FIG. 7 contemplates a patient's gown to be wrapped around a patient and provide forced air warming whilst the patient is transparent to the clinician, anaesthetist or nurse at all times.

A reference to any prior art in this Specification is not, and should not be taken as, an acknowledgment or any form or suggestion that the prior art forms part of the common general knowledge.

1-16. (canceled)

17. A blanket for forced air warming of a patient, the blanket including:
a non-air permeable transparent outer layer;
an air permeable transparent inner layer contactable to the patient;
the outer layer and inner layer being joined at the periphery of the inner layer to define an inflatable chamber, the chamber allowing egress of the forced warming air through the inner layer;
wherein in use, the inflated chamber provides visual transparency of the patient.

18. The blanket according to claim 17, wherein the outer layer and the inner layer are both constructed from biaxially orientated polypropylene film.

19. The blanket according to claim 17, wherein the outer layer and the inner layer are both constructed from any one or more of the following: biaxially orientated polypropylene film; polypropylene film; polyvinyl chloride; and other transparent medical grade plastic.

20. The blanket according to claim 17, wherein the inner layer includes an array of perforations.

21. The blanket according to claim 17, wherein the outer layer and inner layer are integrally formed.

22. The blanket according to claim 17, wherein the outer layer and inner layer are thermally or ultrasonically welded together.

23. The blanket according to claim 17, wherein the inflatable chamber includes a plurality of interconnected compartments.

24. The blanket according to claim 23, wherein each one of the interconnected compartments are disposed in parallel.

25. The blanket according to claim 23, wherein each one of the interconnected compartments are fluted.

26. The blanket according to claim 23, wherein each one of the interconnected compartments are disposed substantially coaxially with any one of the following: an arm of the patient; a leg of the patient; and a torso of the patient.

27. The blanket according to claim 23, wherein each one of the interconnected compartments are defined by thermally welding or ultrasonically welding together a portion of the outer layer and corresponding portion of the inner layer.

28. The blanket according to claim 17, further including an inlet port for receiving the forced warming air into the inflatable chamber.

29. The blanket according to claim 17, including at least one first tie for securing to a forced air warming machine.

30. The blanket according to claim 17, including a plurality of second ties for securing to the patient.

31. The blanket according to claim 17, wherein the use is a single use.

32. The blanket according to claim 17, wherein the blanket includes any one or more selected from the following group: a full-bodied blanket; a lower bodied blanket; an upper body arms-out blanket; an upper body arms-in blanket; a paediatric lower body blanket; a cardiac access blanket; an abdominal access blanket; a multi-access blanket; a surgical access-arms in blanket; and a lithotomy and laparoscopy blanket.
